(19) United States
(12) Patent Application Publication
(10) Pub. No.: US 2023/0011734 A1
(43) Pub. Date: Jan. 12, 2023

(54) DEVICES AND SYSTEMS FOR IMPROVING STENT PERFORMANCE

(71) Applicant: DP Holding (UK) Limited, Grantham, Lincolnshire (GB)

(72) Inventors: Peter Kenneth BALMFORTH, Oslo (NO); Darren SPENCER, Oxford (GB); Paul SOBOTKA, Minneapolis, MN (US); Rodney BRENNEMAN, San Diego, CA (US)

(21) Appl. No.: 17/782,624

(22) PCT Filed: Dec. 4, 2020
(86) PCT No.: PCT/US2020/063450

§ 371 Date: Jun. 3, 2022

Related U.S. Application Data
(60) Provisional application No. 62/944,466, filed on Dec. 6, 2019.

(51) Int. Cl. A61F 2/852; A61F 2/88; A61F 2/95

(52) U.S. Cl. CPC A61F 2/852; A61F 2/88; A61F 2/9517; A61F 2250/0063; A61F 2230/0006; A61F 2230/0008; A61F 2002/9665

(57) ABSTRACT

A stent system is provided comprising a primary stent for location in a lumen of a target vessel, such as a vein or artery that may be fully or partially occluded. The primary stent contacts a vessel wall and at least one secondary stent element is deployed wholly within the primary stent and configured to engage with the interior surface of the primary stent. The secondary stent element is configured to apply a chronic outward radial force to the interior surface of the primary stent so as to effect modification of or to resist change to an aspect ratio of the lumen of the target vessel at the location where the secondary stent element is deployed. In this way the secondary stent element cooperates with the primary stent to restore patency to the target vessel. Various configurations of the stent system are provided as well as deployment devices and methods of treating fully or partially occluded vessels.

### TECHNICAL FIELD

The present invention relates to devices for implantation within the body for improving vessel or duct patency and stent performance as well as for delivery and/or deployment of such devices in the venous system and/or the arterial system.

### BACKGROUND

Coronary stenting and most other stenting situations in the human body occur in relatively stable environments with limited flexure, allowing for stents to be designed to address the specific challenge posed by the environment without too much concern for the flexibility of the stent or its resistance to kinking.

With the development of venous stenting, a number of challenges have been encountered associated with the large capacity and variability of the venous system compared to the arterial system; vein diameter and shape varies highly across the venous system as well as within a vein. The challenges associated with the variability of the venous system are multiplied by the pelvic anatomy, for both arterial and venous interventions due to the high degree of mobility and vessel movement that occurs within this defined zone. This is compounded by the variability of the interaction and location of the pelvic arteries and veins relative to one another and the fixed ligaments and bony protrusions of the pelvis and spine, making the design of an all-purpose “one size fits all” approach highly complex. Variations between men and women as well as between individuals can be quite considerable also. Indeed, current approaches for pelvic vessel stenting necessitates the use of multiple stents of variable design, material, manufacturer, delivery system to try to “replicate” normal anatomy with each stent overlapping each other to accommodate for “slippage” and flex whilst maintaining a continuous apposition to the vessel wall. This results in clinicians often having to adopt a case-by-case approach with solutions patched together from the resources available at the time. Another issue is the lack of predictability for stent placement for extrinsic venous compression. That is, an artery impinging upon a vein requires that a venous stent apply a certain amount of force to displace the artery and restore a near normal venous cross-sectional flow aspect ratio (i.e. closer to unity which is more optimal). Current practice is to place the selected stent hoping for some clinical improvement but little predictability or good options to adjust or improve the outcome after the index venous stent is placed. The net result is that procedures are unnecessarily long or complex, lack predictability or adjustability and can result in post-operative complications.

For example, in U.S. Pat. Nos. 9,192,491 and 8,636,791, modular stent systems are discussed for deployment in the venous system. These systems make use of multiple, overlapping and connected stent to provide differing properties. To facilitate the joining of the two stents, a sealing collar, sometimes in the form of a helical element, is deployed in the region of overlap between the two stents, purely to aid with sealing and joining the two adjacent stents together. While modular stent systems such as this do provide variable properties, they are still prone to slippage and the layering of multiple stents and helical elements may lead to a reduction in luminal diameter. Creating an unnatural obstruction to the flow of blood through the stent may in turn cause turbulence as well as a range of further complications.

It is an object of the invention to provide devices that address at least some of the disadvantages associated with the prior art, particularly in providing improved configurability, restoration and maintenance of vessel patency with minimal complexity.

### SUMMARY

The present invention provides in a first aspect . . . .

A first aspect of the invention provides for a stent system comprising:
a primary stent for location in a lumen of a target vessel, the primary stent defining an exterior surface that contacts a vessel wall and an interior surface that faces inwardly;
at least one secondary stent element deployable wholly within the primary stent and configured to engage with the interior surface of the primary stent,

wherein the at least one secondary stent element is configured to apply a chronic outward radial force to the interior surface of the primary stent so as to effect modification of or to resist change to an aspect ratio of the lumen of the target vessel at the location where the secondary stent element is deployed.

A second aspect of the invention provides for a stent system for restoring patency to a fully or partially occluded target vessel within the body of a subject, the system comprising:
a primary stent for location in a lumen of the target vessel, the primary stent defining an exterior surface that contacts a vessel wall and an interior surface that faces inwardly;
a plurality of secondary stent elements deployable wholly within the primary stent and configured to engage with the interior surface of the primary stent,

wherein the plurality of secondary stent elements are configured to apply a chronic outward radial force to the interior surface of the primary stent so as to effect modification of an aspect ratio of the lumen of the target vessel at the location where the secondary stent elements are deployed.

A third aspect of the invention provides a percutaneous device for deploying a stent element within a vessel located within an individual subject, the device being of elongate configuration having a proximal end and a distal end, the device comprising:
a handle located at the proximal end for mediating control of the device and deployment of the stent element by a user;
a catheter body that extends to the distal end of the device, the catheter body defining and encompassing a central lumen; and
a stent element carrier located at the distal end of the device, the stent element carrier comprising,
an elongate, cylindrical core around which at least one wire for forming the stent element is placed, the core comprising a proximal releasable anchor point and a
distal releasable anchor point, and wherein the at least one wire extends between and is anchored to the proximal and distal releasable anchor points; and
a slidable outer sheath capable of being retracted proximally;

wherein at least one of the proximal and distal releasable anchor points is configured to be movable relative to the other along the longitudinal axis of the device.

A fourth aspect of the invention provides a stent element comprising:
at least one wire formed in a spiral with both ends biased outwardly from a central axis of the spiral;
the outwardly biased ends configured to engage with spaces between the wires of a previously placed braided stent in order to,
(i) anchor the stent element to prevent longitudinal migration and,
(ii) prevent additional circumferential expansion of the stent element and,
(iii) to resist circumferential collapse of the stent element.

A fifth aspect of the invention provides a method of treating an occlusion of a vessel or duct within the body of a subject, the method comprising:
(a) deploying a primary stent within the occluded vessel at a location that spans the occlusion;
(b) deploying at least one secondary stent element within the primary stent so that the secondary stent element applies a radial chronic outward force upon the primary stent thereby relieving the occlusion and restoring patency to the vessel or duct.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### DRAWINGS

One or more embodiments of the invention will now be described, by way of example only, with reference to the accompanying drawings, in which:

FIG. 1 shows a schematic side view of a blood vessel with a stent and an element according to an embodiment of the invention;

FIG. 2 shows the key stent features to be considered in stent design, including chronic outward force, crush resistance and radial resistive force;

FIGS. 3A and 3B show two schematic side views of stents and elements according to embodiments of the invention;

FIG. 4 shows a schematic front view of an element according to an embodiment of the invention;

FIGS. 5 to 7 also show schematic perspective views of elements according to embodiments of the invention;

FIGS. 8A and 8B show schematic front views of elements incorporating spring sections according to embodiments of the invention;

FIGS. 9 and 10 show schematic front views of elements incorporating spring sections according to embodiments of the invention;

FIGS. 11A to 11D show a sequence for inserting a stent system into a vessel according to an embodiment of the invention;

FIGS. 12A to 12H show schematic representations of a device of one embodiment of the invention, foreshortened for ease of representation, in a sequence for creating a stent element;

FIGS. 13A to 13D show a sequence for creating a stent element with a locking mechanism according to an embodiment of the invention;

FIG. 14 shows a diagram of a stent system according to an embodiment of the invention in use, with a primary stent located adjacent to a compressive occlusion and a stent element deployed within the primary stent to exert a radial chronic outward force at the site of occlusion to restore patency;

FIGS. 15A to 15D show different mechanisms for positioning the stent element relative to the primary stent, (A) shows the presence of a plurality of radiopaque markers spaced longitudinally to ensure correct positioning and alignment; (B) shows ultrasound windows to allow IVUS visualization during stent element deployment; (C) shows a radiopaque flexible nose cone at the distal terminus; and (D) shows the over-the-wire configuration with a guide wire indicated as passing through a central lumen of the device;

FIGS. 16A to 16C show (A) simultaneous arterial and venous contrast injection in a therapy resistant hypertensive patient, with no signs or symptoms of leg swelling (LAO orientation). (B) and (C) demonstrate impeded contrast flow in the vein via direct overriding arterial compression taken from both AP and LAO angles respectively; white arrows show the location of the venous obstruction; and

FIG. 17 shows a photograph of a ribbon coil stent element according to one embodiment of the present, an aperture suitable for engagement with a pin release mechanism on a deployment device is visible.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term ‘braided stent’ refers to a metal or metal alloy stent that is produced using a plain weaving technique. The stent comprises a lumen capable of stretching in the longitudinal direction while circumferentially, the multiplicity of filament-like elements intersect a plane that is perpendicular to the longitudinal direction when in the expanded position.

The term ‘kink resistance’ refers to a stent's ability to withstand mechanical loads from the surroundings depending upon the position in the body. Usually, this is based upon the smallest radius of curvature a stent can withstand without the formation of a kink. In areas of high tortuosity within the body it is necessary for a stent to have increased kink resistance to prevent a reduction in lumen patency or even total occlusion.

The term ‘crush resistance’ refers to the ability of a stent experiencing external, focal or distributed loads to resist collapse. These loads ultimately lead to stent deformation and even full or partial occlusion which can result in adverse clinical consequences.

The term ‘venous ulcers’ refers to skin sores that form due to the persistent elevation of venous pressure. Often, they present in association with venous valve regurgitation. They are most commonly found on the lower limbs. It is thought that when venous valves become mechanically blocked or veins become engorged and the valve leaflets cannot co-opt to prevent regurgitation of blood, venous congestion worsens and the hydrostatic forces cause both extravasation of fluid from the veins into interstitium, and activation of inflammatory cytokines. This accumulation of fluid pressure and inflammatory cytokines contributes to skin break down, chronic ulceration and predisposes to local infections.

The term ‘venous obstruction’ refers to any occurrence whereby the diameter (or ‘caliber’) of a vein is reduced when compared to a normal, i.e. non-occluded, state. Venous obstruction can occur through the narrowing (stenosis) of the vein, through blockage or through externally applied pressure causing a localised compression of the vein. The term also includes venous occlusion, whereby the vein's lumen is partially or totally obstructed to the flow of blood. Occlusion may result from thrombosis (e.g. deep vein thrombosis (DVT)) or may be due to tumour incursion.

The term ‘venous return’ is defined by the volume of blood returning to the heart via the venous system, and is driven by the pressure gradient between the mean systemic pressure in the peripheral venous system and the mean right atrial pressure of the heart. This venous return determines the degree of stretch of heart muscle during filling, preload and is a major determinant of cardiac stroke volume.

The term ‘venous compression’ refers to the external compression of the vein. The source of external compression may be caused by an adjacently located artery compressing the vein against another fixed anatomical structure, which can include the bony or ligamentous structures found in the pelvis, the spine itself, or overlapping arterial branches.

The term ‘May-Thurner syndrome’ (MTS) also known as iliac venous compression syndrome (which includes Cockett's syndrome) is a form of ilio-caval venous compression wherein the left common iliac vein is compressed between the overlying right common iliac artery anteriorly and the lumbosacral spine posteriorly (fifth lumbar vertebra). Compression of the iliac vein may cause a myriad of adverse effects, including, but not limited to discomfort, swelling and pain. Other less common variations of May-Thurner syndrome have been described such as compression of the right common iliac vein by the right common iliac artery; this is known as Cockett's syndrome. More recently, the definition of May-Thurner syndrome has been expanded to include an array of compression disorders associated with discomfort, leg swelling and pain, without the manifestation of a thrombus. Collectively, this has been termed non-thrombotic iliac vein lesions (NIVL).

The term ‘infraluminal thickening’ (also referred to as venous spurs or intraluminal spurs) is related to this external compression of the left common iliac vein by the right common iliac artery against the fifth lumbar vertebra. Venous spurs arise due to the chronic pulsation of the right common iliac artery, this ultimately results in an obstruction to venous outflow. Venous spurs are internal venous obstructions consequent to chronic external compression of veins by adjacent structures.

The term ‘Deep Vein Thrombosis’ (DVT) refers to the formation of blood clots or thrombus within the venous segment, and in itself is not life threatening. However, it may result in life threatening conditions (such as pulmonary embolism) if the thrombus were to be dislodged and embolize to the lungs. Additionally, DVT may lead to loss of venous valvular integrity, life long venous incompetence and deep venous syndrome which includes rest and exercise pain, leg swelling and recurrent risk of DVT and emboli. The following is a non-limiting list of factors that reflect a higher risk of developing DVT including prolonged inactivity, smoking, being dehydrated, being over 60, undergoing cancer treatment and having inflammatory conditions. Anticoagulation which prevents further coagulation but does not act directly on existing clots, is the standard treatment for deep vein thrombosis. Other potentially adjunct, therapies/treatments may include compression stocking, selective movement and/or stretching, inferior vena cava filters, thrombolysis and thrombectomy.

Stents were first designed for use in the cardiovascular space in the mid-1980s and have since undergone major refinements in design and composition. The indications for stenting and locations of their use in the human body has also developed; stenting of arterial and venous vessels is a regular occurrence in hospitals.

One of the original stents, the Wallstent (Schneider AG), was a self-expanding, stainless steel wire-mesh structure. This was superseded by the Palmaz-Schatz stent (Johnson & Johnson), which was the first FDA approved, balloon-expandable stainless steel slotted tube stent. Multiple stents and stent manufacturers followed shortly after with their own iterations that were designed to prevent elastic recoil and restenosis. These were far from optimal stent designs because they had a high metallic density that resulted in elevated rates of stent thrombosis, failed deployments, embolizations and in-stent restenosis (ISR). For example, restenosis occurred in 20% to 30% of all angioplasties.

Drug-eluting stents (DES) were developed to specifically address the issues of ISR. Seen as the next revolution in interventional cardiology, DES utilized a coating of various compounds to target proliferation of vascular smooth muscle cells, platelet activation, and thrombosis. Many compounds were tried with minimal response, including gold, carbon, heparin, and others such as oestrogen, glucocorticoids, and mineralocorticoids with modest effects. However, the greatest effect was seen in the use of anti-proliferative drugs. Drugs such as Sirolimus and Paclitaxel were the most effective in reducing ISR.

This lead to a new generation of stents, stent design, and stent coating combinations. Early signals were very positive, indicating better efficacy when compared with bare metal stents. However in 2006, a safety signal began to emerge of an increased risk of stent thrombosis (ST) in first generation DES. A redesign of the first generation DES lead to a second generation of DES with novel antiplatelet agents and polymers.

While stents were being designed in an attempt to counteract ST, the physical implantation of a stent itself acted as the perfect recipe for thrombus formation, and required the use of complex anticoagulation regimens to combat ST. This caused further problems, leading to major bleeding and vascular complications in many cases. It wasn't until the development of dual antiplatelet therapy (DAPT) that stents began to become safer to use in common practice.

Stent materials and designs have continued to be developed over the years. First generation stent materials such as stainless steel have been more recently superseded by cobalt-chromium alloys. Cobalt-chromium alloys allow for thinner stent strut designs without compromising radial strength or corrosion resistance of the stent. Other new alloys include platinum-chromium alloys, which are used for high conformability and radial strength and a thinner stent strut design.

In addition to drug coatings and drug developments, stents have also been covered with various synthetic or biological materials in order to cover perforations, aneurysms, or heavy thrombus. Bioresorbable stent scaffolds have also been designed to provide a vascular scaffold following a percutaneous coronary intervention (PCI). The bioresorbable scaffolds are gradually re-absorbed after placement, leaving the vessel in which the scaffold was placed free from any metallic caging and able to regain its normal function. A number of biodegradable compounds have been developed and utilized by various manufacturers for this purpose.

In today's stent market there exist five different types of available stents:
Dual therapy Stents (DTS)
Bioresorbable Vascular Scaffolds (BVS)
Bio-engineered Stents
Drug Eluting Stents (DES)
Bare Metal Stents (BMS)

Their overall main purpose is to keep narrowed blood vessels open to allow adequate flow of blood or other bodily fluid. A special group of stents, called stent grafts are used in the aorta to create a smaller conduit within which the blood can flow, as the original vessel has become enlarged and at risk of rupture. Various applications of stents in the body include:
Coronary Stents
Urinary stents
Urethral and Prostatic stents
Peripheral vascular stents
Stent grafts
Oesophageal stents
Biliary stents

Hence, embodiments of the devices according to the invention can also be used during endoscopic and laparoscopic procedures where the vessel includes the bile duct, the intestine, the fallopian tubes, the ureter, the urethra, the oesophagus, bronchioles, or any other hollow vessel or duct within the body of an animal.

Venous stents require unique characteristics that differ from arterial stents. Veins are highly flexible and vary in diameter and luminal profile depending upon flow and surrounding structures that may impinge upon them. Veins operate at very low pressures, relative to arteries, therefore it is critical that they are able to expand to accommodate additional flow during exertion. Venous stents must likewise be self-expanding, flexible and adapt to the changing nature of the veins in which they are placed. Venous walls are prone to deformation due to normal movements such as the overlying musculature, organ function (e.g. peristalsis), as well as the respiratory and cardiac cycle. At the same time, venous stents are placed because there is some obstruction or external compression to be resisted, so they must have appropriate strength to restore luminal flow diameter at the treatment site. Of course, once a stent is implanted the walls of the vein will react to the deformation inherently caused by the device. The interplay of the stent and the externally applied forces may vary along the length of the stent resulting in irregular mechanical interactions along the longitudinal axis. These irregularities can result in stent migration and associated complications.

Despite vast improvement in stent design since the origination of stenting, the stent options currently available on the market are plagued by a number of problems including foreshortening, device collapse, device failure, device wear and eventual perforation. Some of the main underlying factors contributing to the problems with these stents include a lack of flexibility or too much flexibility. Increased load on the deformation of the stent can cause early fatigue failure, and/or impedance of flow in the overlying iliac artery, potentially causing peripheral arterial disease.

An underestimated but important problem experienced by stents is stent fracture. The incidence of stent fractures for coronary stents is on average around 4%. Many stent fractures may not be recorded as a number of fractures are likely asymptomatic without any sequalae. Alternatively the fractures may not be detected with conventional angiography procedures, and so are diagnosed and treated otherwise, perhaps as stent thrombosis or stenosis rather than stent fracture. Finally fractures may lead to stent thrombosis. In such cases, the first presence of a fracture may be the onset of sudden cardiac death. Again, such fractures are not recorded. Stent fractures have been identified to be more likely to occur as a result of one or more of the following:
balloon or stent overexpansion;
stent overlap creating localized rigidity;
stent length: long stents with high radial forces;
inappropriate stent handling;
poor stenting technique;
a lack of stent conformability (conformability being how far the stent can bend around its axis);
tortuous highly angulated vessels;
long lesions;
change in vessel angulation post implantation;
complex lesions; or
inappropriate stent locations.

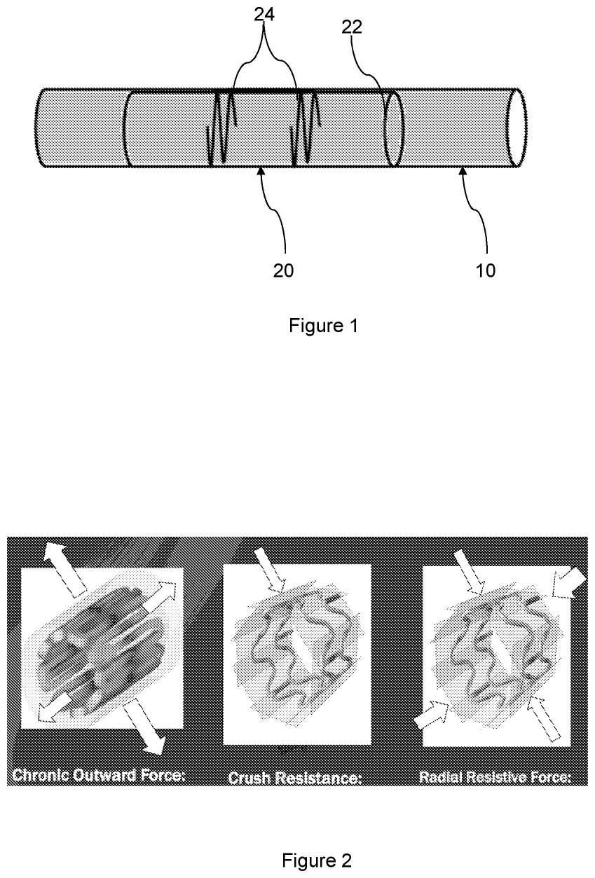

FIG. 1 shows a schematic representation of a blood vessel 10 incorporating a stent system 20 according to an embodiment of the invention. The blood vessel 10 may be an artery or a vein, or even a non-vascular duct. The stent system 20 comprises a stent 22 positioned within the lumen of the vessel 10 and in direct contact with the tissue forming the vessel 10 when the stent 22 is deployed. The stent 22, which may be referred to as a ‘base stent’, ‘main stent’, or ‘primary stent’, is any stent considered suitable for placement in the lumen of the vessel 10. For example, the stent 22 may be one of the five types of presently available stents listed above. However, while conventional practices may dictate that the stent 22 should be highly adapted for the purpose, by changing the properties of the stent through its design, in the present embodiment, the stent 22 may be any suitable stent and so may, in some embodiments, be a stent of relatively generic design. In specific embodiments of the invention, the stent 22 may already be in situ—i.e. it is a previously implanted stent.

In general, the primary stent 22 is any stent suitable for the application it is being used on. The primary stent 22 may be a braided stent, a laser cut tubular stent, or another suitable stent. The primary stent 22 may have a minimum radial resistive force, kink resistance, and/or crush resistance as appropriate, which can subsequently be augmented with stent elements. The primary stent 22 can be manufactured from any suitable material. The primary stent 22 may have any suitable dimensions relative to the vessel in which it is located. The primary stent 22 may have any suitable covering, and/or any suitable drug coating if required. The primary stent 22 may have specific or non-specific properties for the application for which it is being used. The primary stent 22 may be constructed from a variety of different strengths of wire/different weave structures specific to the location of deployment.

In some examples, the primary stent 22 may have the same mechanical properties across the entire primary stent, or may have variable mechanical properties, having defined regions of low crush resistance or flexibility and other regions of high crush resistance for example. In some examples of embodiments, the primary stent may be fully or partially tapered or exhibit a graduated luminal diameter from one terminus to the other. Some examples of primary stents may have specific locations for side apertures to prevent thrombus formation or allow formation of anastomoses to adjacent vessels. In embodiments of the invention the stent 22 may include one or more junctions, bifurcations, or anchoring sections that allow the stent to conform to local vessel anatomy,

In pursuing a more personalised or customized stent design, the inventors have identified that the design of stents itself can only be optimised to an individual patient's anatomy to a certain extent, and that changes in the vasculature of patients may result in the off-the-shelf design of stent being unsuitable for the vasculature. It is also very difficult for physicians currently to assess the potential success for a given stent to adequately restore luminal diameter. It is only after placement of the chosen stent that a physician may realize that insufficient luminal diameter has been restored, resulting in an ovalized or high aspect ratio lumen, or insufficient force to resist the external compression with no good options for correction or adjustment. Accordingly, the inventors have provided a configurable stenting system 20 having the primary stent 22 and, within the primary stent 22, at least one secondary stent 24, referred to hereafter as a ‘stent element’, that provides a localised change in the physical and/or mechanical properties of the primary stent 22. In embodiments of the invention, the stent element 24 is not in direct physical contact with the endothelial tissue of the vessel 10 when disposed in the lumen of the primary stent 22. Rather, the stent element 24, when deployed, bears upon the inner luminal surface of the stent 22.

The primary stent 22 may be designed in tandem with the stent element 24 to enable connection between the stent elements 24 and the primary stent 22. For example, as will be described later, the system 20 may comprise an anchoring mechanism for mediating an interconnection between the stent 22 with the one or more stent elements 24.

As shown in FIG. 1, in this embodiment two stent elements 24 are provided within the lumen of the primary stent 22. The stent elements 24 are provided for deployment within the lumen of the primary stent 22, and have an aperture or central lumen through which blood may flow unobstructed along the vessel 10, the aperture being defined by an inner-facing surface of the elements. An outer-facing surface of the stent elements 24 engages with the interior-facing luminal wall of the primary stent 22. The stent elements 24 are configured to be positioned and to maintain that position within the primary stent 22 by an engagement between the outer surface of the elements and the interior of the primary stent 22. The engagement may be mediated via an anchor mechanism, or by simply by friction generated by application of an outward radial force, or by other means (e.g. spot welds, use of adhesive compounds etc.).

The stent element 24 is configured to provide reinforcement to the primary stent 22. As shown in FIG. 2, the reinforcement may be provided by providing, at the location of the stent elements 24 within the primary stent 22, an improvement of one or more of stent properties selected from the following: chronic outward force, crush resistance, kink resistance (not depicted in FIG. 2), and/or radial resistive force. It is desirable for the stent elements 24 to have a balance of these key properties relative to its placement within the vessel 10.

As can be seen in FIG. 2, chronic outward force is a radial force that a stent element exerts at its placement location. Crush resistance, as defined above, is a stent's ability to resist collapse under focal compression. Radial resistive force occurs under concentric compression and is related to the stent's ability to resist collapse under this type of compression. The kink resistance of a stent is its ability to withstand forces due to movement of the body and its ability to be kinked without deformation of the stent. In providing reinforcement, the stent element 24 may act to bring the aspect ratio of the primary stent 22 and, in so doing the vessel itself closer to a value of one (i.e. unity) so that the lumen of the vessel and primary stent 22 are substantially circular in cross section at the position of the stent element 24, and, preferably, along the length of the rest of the primary stent 22. As discussed above, a significant cause of stent failure is due to a lack of sufficient radial force necessary to overcome the venous or arterial obstruction that they are deployed to counteract. The stent element 24 is provided to introduce a localised radial and/or compressive force to the primary stent to better counteract an obstruction. Importantly, the inventors have identified that obstructed vessels may often have non-circular lumens, and therefore exhibit an aspect ratio of less than or greater than one, depending upon which aspects are considered. Accordingly, primary stents may also have the same aspect ratio. Deploying a stent element 24 to restore or impose an aspect ratio close to one acts to apply the necessary forces to the obstruction and thereby restore optimal patency to the vessel.

As the stent elements 24 are configured to restore or impose an optimal aspect ratio to the vessel 10 and/or the primary stent 22, the stent element 24 may be formed to have a substantially circular cross section/outline, or it may be formed to have a different polygonal cross-section or outline. For example, an elliptically shaped stent element may provide a useful counteraction to an elliptically shaped vessel. Depending upon the application, in certain embodiments the stent element may be shaped as a polygon in cross section, such as a hexagon. In such embodiments the outward radial or compressive forces are exerted at foci located at the corners of the polygon as opposed to the more even distribution of force associated with a stent element 24 having a circular configuration. Alternatively or additionally, the stent element may have a non-linear cross-section, either being tapered and/or having twists/rotations and/or comprising a combination of shapes to appropriately tailor the required radial forces to the vessel to counteract the obstruction.

Providing one or more stent elements 24 for deployment in the primary stent 22 permits a different approach to conventional stenting. Historically, as discussed above, stents have been increasingly refined in their design for highly specific applications, and as a result compromises and modifications have been made to mitigate the issues introduced by the increasing complexities.

In providing the stenting system 10 in the form of a kit of the primary stent 22 and one, two, three, four or more independently positionable stent elements 24, the complexities and associated issues of highly-specialised stent design are avoided. For one, the primary stent 22 can be a stent without a tailored design, and so can be flexible and straightforward to position and maintain. The primary stent 22 can be considered to be less intrusive and obtrusive. A reduction in the complexity of the primary stent 22 lends itself to an improved ease of manufacture and reduction in cost of production as well as associated lower burden for regulatory approval also.

Furthermore, the reduction in complexity is preferable for the increase in the use of stents. As human anatomy can be variable between individuals, the design of stents for use in many patients is difficult as the exact requirements for regions of different flexibility are unlikely to be the same between any two patients. For example, pelvic venous anatomy of juveniles versus geriatric patients, or even men versus women, can be quite different. In veterinary contexts, the divergencies between various sub-species of animal, such as breeds of dogs, is also highly variable. Accordingly, the use of a flexible primary stent 22 and one or more implantable stent elements 24 allows for a faster, more personalised stent system 20.

In embodiments of the invention, the one or more stent elements 24 can be arranged within the primary stent 22, either before or after insertion, to quickly provide a personalised stent arrangement with regions having different properties. The properties are also more variable because the elements can be designed accordingly. These reinforcing stent elements 24 can be positioned exactly where changes in radial force and/or crush resistance and/or kink resistance are desired, whilst minimizing the compromise of the primary stent 22 property of flexibility in mobile regions of the body, such as the pelvic geometry.

Further benefit is provided by the elements because they are implantable after the primary stent has been inserted, and their position is variable and can be changed if the vessel's properties also change. In providing implantable stent elements, remedial action intended to address complications with existing pre-inserted stents may also benefit from the changes in mechanical properties that result from using these implantable elements. In this way the primary stent pre-implanted stent can be maintained within the vessel and invasive procedures to remove the stent are avoided.

Put simply, the stenting system 20 described herein can provide similar benefits as existing, more exotic specialised stents, but without the disadvantages associated with existing stents such as the cost, complexity of design, failure rate or the difficulties of insertion and deployment.

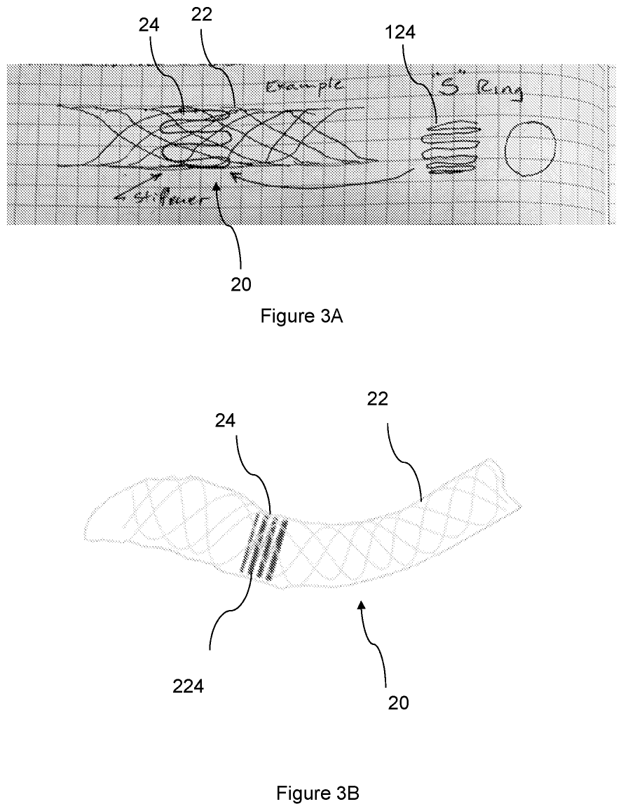

Typically, the stent elements 24 are, as described above, secondary stents for placement wholly within and encompassed by the primary stent. Therefore, the stent elements 24 may be formed as a stent by any suitable means and in by any appropriate method. In a specific embodiment, both the stent element 24 and the primary stent 22 may be comprised of braided wire. The stent element 24 may be formed of one or more wires, arranged to provide the optimal radial force and in the desired shape to restore the required aspect ratio of the stent and surrounding vessel. Hence, the wire may be formed of any appropriate material, in any cross-section, to provide the desired effect. Different shapes of wire in cross section (e.g. round, elliptical, hexagonal, square and/or rectangular) allows for different design characteristics in the stent element. For instance, a flat rectangular wire or ribbon, may allow for better engagement with the primary stent than a round wire which may slip. Oval wires may allow for added strength without increasing the overall thickness of the device. Two stent system embodiments 20 including specific types of stent element 24 formed of wire are shown in FIGS. 3A and 3B. In each figure, a primary stent 22 is shown in the form of a braided stent. A ribbon coil stent element according to one embodiment is shown in FIG. 17.

According to a specific embodiment of the invention, the stent element 24 as shown in FIG. 3A, is in the form of an “S” ring 124. The S ring 124 is a loop of wire, in which the wire is arranged to curve back on itself in a zig-zag pattern to create an S shape that is repeated around the circumference of the reinforcing stent element so as to form a cylindrical collar. In FIG. 3, the S shape takes the form of sections that are parallel with one another and the longitudinal axis of the stent element, joined by curved sections. The properties of S ring 124, such as the thickness and material of the wire, the length of the reinforcing element, the acuteness of the curves in forming each S shape, and how tightly the curved parts are packed together are variable to vary the properties of the stent element. Accordingly, the radial resistive force, crush resistance, and kink resistance may be optimized for the application. The S ring 124 is deployed within a primary stent and maintains placement therein through recoil force applying an outward radial pressure on the primary stent. In this way the stent element 124 maintains an outward force that restores patency to the stent and, thus, the vessel. By varying the distance between parallel parts of the S-ring 124, the shape of S-ring 124 may be altered to vary the aspect ratio. The stent element 124 may be comprised of a material such as a shape memory alloy that allows for compression during delivery, followed by self-expansion upon deployment, such as via an intraluminal catheter delivery system.

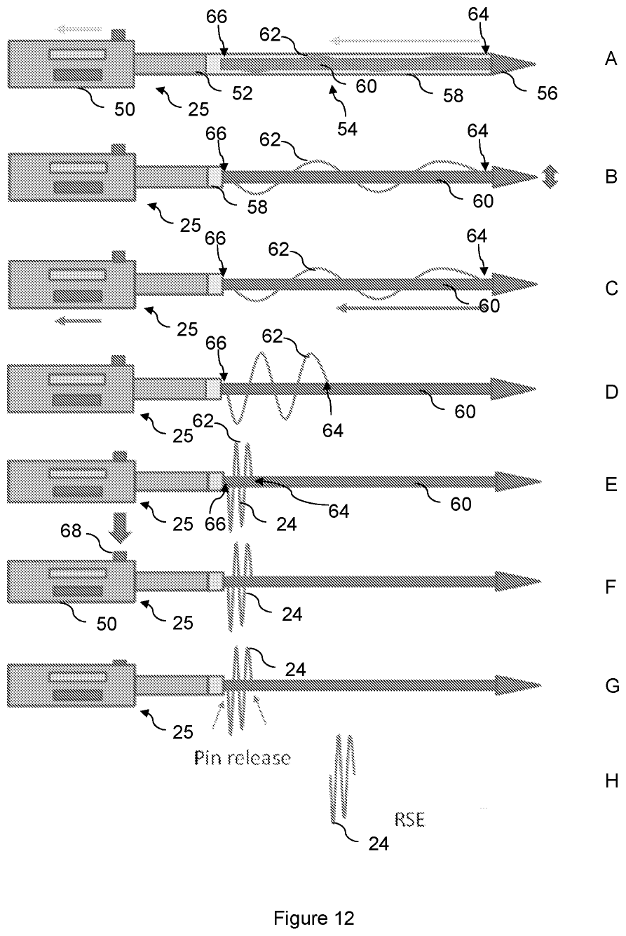

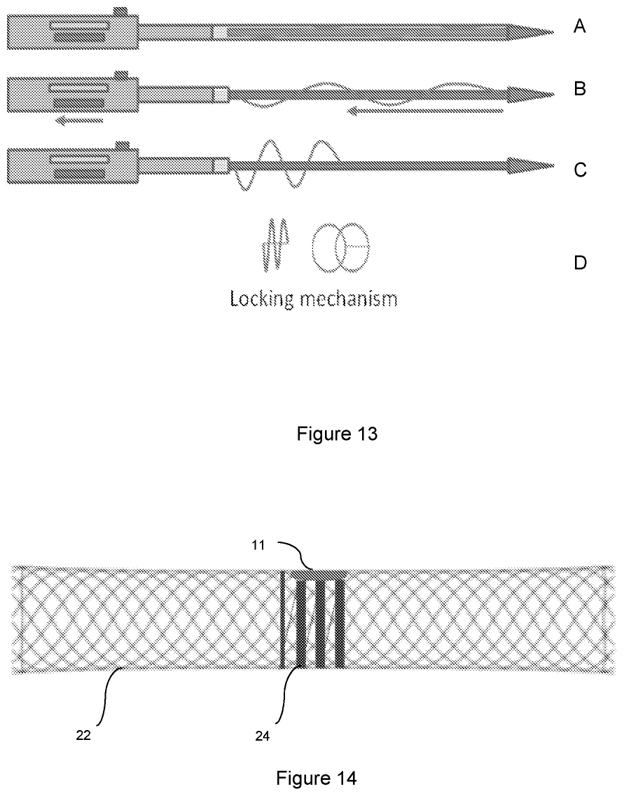

In the embodiment depicted in FIG. 3B, the stent element 24 is a coil element 224. The coil 224 is suitably a coil of wire formed to have circumferential dimensions similar to the interior of the primary stent 22 when fully expanded upon deployment. The number of coil turns, the length of the stent element, the distance between the coil turns, the material from which the coils are formed, and a number of other properties are variable to optimize the relevant properties for use in the primary stent. The method and apparatus used for deployment of a stent element in the form of a coil element 224 according to one embodiment of the invention is discussed later in relation to FIGS. 12 and 13.

In general, the radial force and crush resistance of the stent element 24 is based on and controllable by varying the thickness and cross sectional shape of the wire, type of wire, construction of the element 24 through twists or braids, and other properties of the wire forming the stent element 24. The properties of the stent element 24 may be controlled by selecting specific braiding patterns or the specific number of coil turns or twists necessary to achieve a desired outward radial force. In embodiments of the invention, stent elements 24 of embodiments of the invention exert an outward radial force of greater than 0.25 N/cm, suitably at least 0.5 N/cm, typically at least 1.0 N/cm and optionally at least 2 N/cm. In further embodiments of the invention, the stent elements 24 exert an outward radial force of at most 25 N/cm, suitably at most 20 N/cm, optionally at most 15 N/cm. In embodiments of the invention the outward radial force is determined at greater than 50% expansion of the stent element, or alternatively at between 10% and 50% of the expansion.

In other examples that are not depicted here, the stent element may comprise material arranged otherwise than a coil 224 or S-ring 124. For example, the stent element 24 may be S-shaped in the circumferential direction so that each S forms one circumference of the element. Another alternative is a double-helix arrangement of two coils whose ends are joined.

In some stent elements 24, a combination of a coil and an S-ring may be provided. For example, the stent element may comprise two S-rings having a coil extending between them, all comprised from a single length of wire.

It is envisaged that in specific embodiments the individual stent elements 24 will be at least 1 mm, suitably 5 mm, optionally 10 mm in length; and at most 30 mm, and typically not more than 20 mm in length.

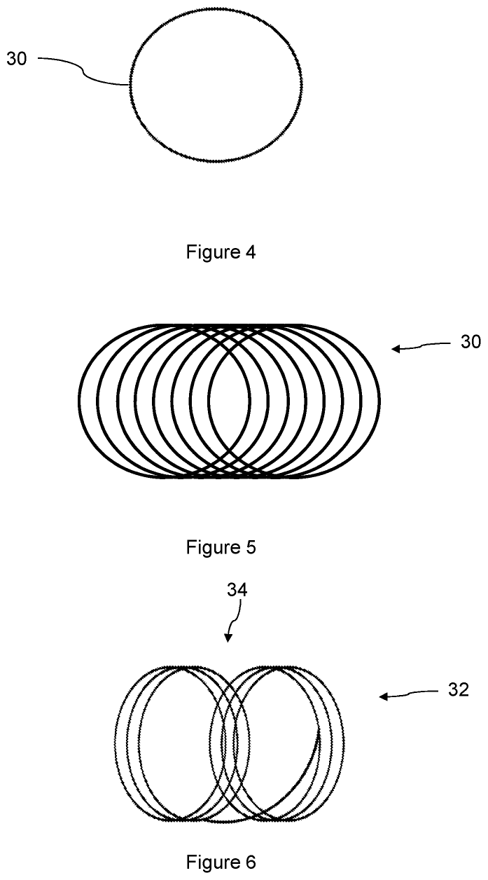

To provide a generalized discussion about the stent elements 24 and the possibilities for their use, FIGS. 4 to 10 are depicted as schematic representations of stent elements 24 each having a main component part 30, as shown in FIG. 4. This component part 30 may each be a single S ring, coil, or otherwise, or may be part of a single S ring, coil, or otherwise. In other words, the use of circles in FIGS. 4 to 10 is for depicting and explaining the general qualities of the stent elements and for clarity only, rather than as an indicator that multiple stent elements or coils or rings are to be placed together. Moreover, the use of circles is for ease of example only, and the embodiments described herein are by no means limited to circular stent elements. As noted above, the stent elements may comprise stent elements having other cross-sections, such as polygons.

FIG. 5 shows an exemplary elongate stent element 24 of one embodiment of the invention having a uniform diameter along its length. The stent element 24 may be used to provide a region of a primary stent with improved properties. For example, the stent element 24 may provide an increased radial force that is the same along the length of the stent element 24.

The element 30 of FIG. 5 may be formed of a single coil, i.e. a single stent element 24, or may be formed of a plurality of interlinked stent elements 24. By providing a single coil, the same properties are provided along the length of the element 30. Interlinking a plurality of stent elements 24 is a particularly useful means for enabling regions of variable properties over a short distance. This is typically something that is more difficult to achieve with conventional stents because the stent's mechanical properties must be provided by the relatively continuous cell structure or braiding. However, by using multiple stent elements 24 linked together, these properties can be varied over relatively short distances, thereby improving the personalization potential of the primary stent to the specific clinical context in the patient.

A variation on the element 30 of FIG. 5 is the element 32 shown in FIG. 6. The element 32 of FIG. 6 is a stent element 24 having two joined parts that are separated by a space 34. The incorporation of a space 34 within a stent element, or between joined stent elements, enables a further variation on the properties of the primary stent. By incorporating a space 34, the properties of the primary stent are maintained while being changed either side of the space 34. The benefit of using a stent element 32 of this form including one or more spaces between joined elements is that one procedure is required to insert a linked stent element, so that the invasiveness of the insertion of the stent elements is reduced.

In some examples, the stent element 32 may be movable so that the space 34 between the parts can be varied to ensure an exact placement of the stent element when inserting. It will be appreciated that a coil formation lends itself to this stent element shown in FIG. 6 because the element of FIG. 6 can be formed using a single wire, but any of the element types discussed herein may also be used to form such a stent element with a space.

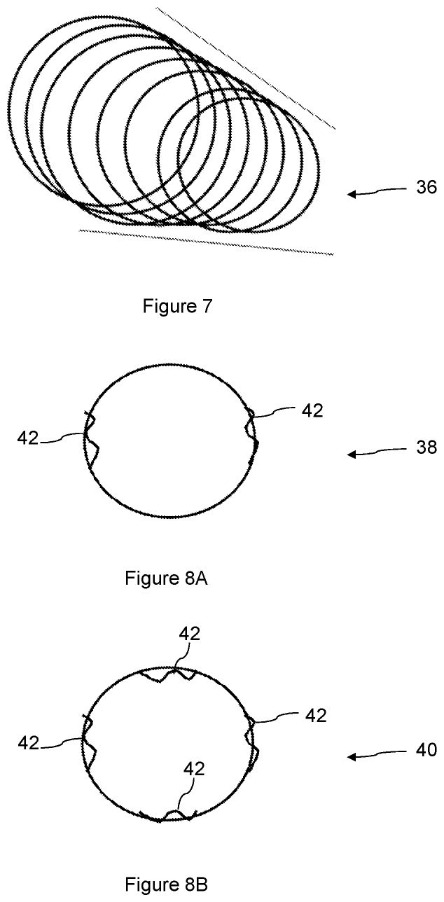

FIG. 7 illustrates a tapered stent element 36. Tapered stent elements 36 may have a diameter that changes towards one end or towards both ends (i.e. from terminus to terminus), and are useful for deployment in tapered primary stents. Tapered stent elements 36 may also be utilized to change the properties of the stent element along its length.

FIGS. 8A and 8B illustrate embodiments of stent elements 38, 40 incorporating springs 42 around their circumferences. In FIG. 8A, two springs 42 are provided on opposing sides of the stent element 38, while in FIG. 8B, four springs 42 are provided, equally spaced around the stent element 40. Springs 42 may be provided anywhere on a stent element, and any number of springs 42 may be incorporated into a stent element to vary its properties accordingly. Crush resistance of the stent element may be based on and controllable by the incorporation of these springs or spring-like portions into the stent element and the biasing properties of these springs themselves.

It will be appreciated that where the term ‘spring’ is used, this is intended to mean an elastic member capable of being compressed or elongated in a direction and returning to its original length. In the examples provided here, the springs are compressible and stretchable in directions that are at a tangent to the circumference of each stent element.

Springs 42 may be incorporated into both coil stent elements and S-ring stent elements as appropriate and stent elements can be combined with or without spring like elements to achieve the desired properties. In particular, any of the arrangements of FIGS. 3A to 7 may incorporate springs to effect a change in the crush resistance of the element. For example, each spiral rotation of a coil stent element may incorporate one or more springs. Spring sections may also be incorporated into the wire of the stent element 24 itself such as via presence of a portion of the wire having a reduced diameter, thereby resulting in an alternation in local mechanical properties.

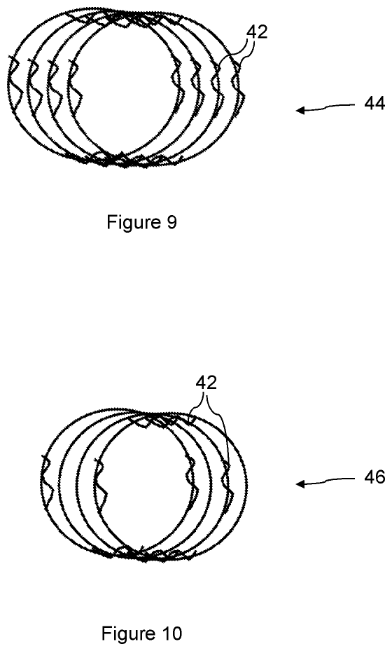

Two embodiments are shown in FIGS. 9 and 10. As shown in FIG. 9, the springs 42 are spaced equally around the circumference of the stent element 44, so that the locations of the springs 42 along the length of the stent element 44 repeats. As shown in FIG. 10, the stent element 46 may incorporate springs 42 that are arranged at different points and whose positions do not repeat regularly. Springs 42 allow the stent element to recoil during movement or added load, without fracturing under that load. In addition, placement of spring sections about the circumference may alter the compression or expansion properties of the stent element within a specific plane or axis.

In each of the above embodiments, the end termini of the wire or material forming the stent element may be free. In other examples, the free ends can be joined or locked together either prior to positioning within the primary stent or once deployment of the stent element within the primary stent is complete. Joining or locking the ends prevents the free ends from perforating the vessel walls or snagging the deployment device or guide wire, adds strength to the ends of the stent elements, adds strength to the entire length of the stent element, and improves the stability of the reinforcing stent element.

Although not depicted in the Figures, the stent elements and/or the primary stent may incorporate one or more physical mechanisms to maintain the relative positions of the stent elements and primary stent. In specific embodiments the retention mechanisms may incorporate one or more hooks, teeth, barbs or splines that engage with or bear upon the primary stent and prevent malposition or subsequent migration of the stent element. In general, it is expected that friction between the stent elements and the primary stent and the outward radial bias force exerted by the stent elements on the interior face of the primary stent will be sufficient to maintain the stent elements in position. However, a further mechanism may be particularly useful to ensure that the relative positions are maintained even through the continual movement and changes in vasculature that occur with everyday activity. The mechanism may anchor the stent element to the primary stent. In some embodiments, the stent element may incorporate the mechanism so that the stent element is attached to and grips the primary stent. In some embodiments, the primary stent may incorporate a mechanism so that the stent element is gripped or anchored by the primary stent. In some embodiments, both the stent element and the primary stent incorporate parts of the mechanism so that there is an interaction between two parts of the mechanism to anchor the stent element to the primary stent.

The stent element may incorporate the anchoring mechanisms to prevent its migration relative to the primary stent. These mechanisms include but are not limited to loops, taps, bends, gripping elements, or surface modifications of various shapes and designs. The anchor mechanisms may be provided at either or both termini of the stent element or anywhere along the length of the stent element.

In a particular embodiment, the stent elements may comprise flexible hooks arranged about the circumference of a coil. Each hook faces the same direction. The stent element can therefore be mounted to and affixed to the primary stent by positioning it and rotating in the direction of the hooks so that they hook onto the primary stent.

In some embodiments, the primary stent may incorporate an engaging mechanism for engaging the stent element and maintaining its position relative to it. Similarly to the anchoring mechanism of the stent element, the engaging mechanism may comprise loops, taps, bends, gripping elements of various shapes and designs. The engaging mechanism may interact with the anchoring mechanism of the stent element or may engage the stent element without a specific anchoring mechanism.

In one example, the engaging mechanism comprises have internally-extending hooks that all face in one direction longitudinally along the stent that are configured to hook onto the wire of the stent element. To engage the stent element, the stent element is maneuvered along the primary stent, and once the correct position for the stent element is reached, it is pulled backwards slightly to engage with the hooks, thereby connecting the stent element to the primary stent.

In one embodiment of the present invention, the primary stent may include one or more coupling elements to prevent migration of the primary stent within the vessel. The coupling elements may be provided at one or both termini of the primary stent.

To aid positioning of the stent elements, radiopaque markers may be provided along the length of the primary stent to indicate relative positions.

The stent element and/or the primary stent may comprise of, either separately or in combination, stainless steel, nitinol, cobalt chromium, tantalum, platinum, tungsten, iron, manganese, molybdenum, or other surgically- and bio-compatible metal or metal alloy.

The stent element and/or the primary stent may comprise non-metal material, including a polymer such as: a bioresorbable material such as poly (l-lactide) (PLLA), polyglycolic acid (PGA), polyglycolic-lactic acid (PLGA), polycaprolactone (PCL), polyorthoesters, polyanhydrides, or another aliphatic polyester fibre material; polypropylene; polyamide; carbon fibre; and glass fibre. In some embodiments, the stent element and/or the primary stent comprise both metal and non-metal portions. The stent element and/or the primary stent may comprise radiopaque markers to assist with optimal placement and orientation longitudinally and/or radially. Such radiopaque material may include titanium, tantalum, rhenium, bismuth, silver, gold, platinum, iridium, and/or tungsten.

The primary stent or portions of the primary stent may be covered. Such covering material may include: PTFE; e-PTFE; polyurethane; silicone; papyrus; Dacron®; Goretex®; other polymeric membrane; polyhedral oligomeric silsesquioxane and poly(carbonate-urea) urethane (POSS-PCU); other Biodegradable nanofibers. The stent element or portions of the stent element may be covered. Covering material may include any of the above-referenced covering materials.

In specific embodiments of the invention, the primary stent may contain a window or cell of increased size and identified by radiopaque markers to allow for the creation of an anastomosis shunting device with an adjacent vessel or duct, without requiring the perforation of the primary stent structure.

The primary stent and/or the stent element may comprise of a drug coating or combination of drug coating and graft covering to promote re-endothelization; improve endothelial function; reduce inflammatory reaction; inhibit neo-intimal hyperplasia (MM2A); prevent adverse events such as in-stent restenosis and stent thrombosis through antithrombotic action of heparin.

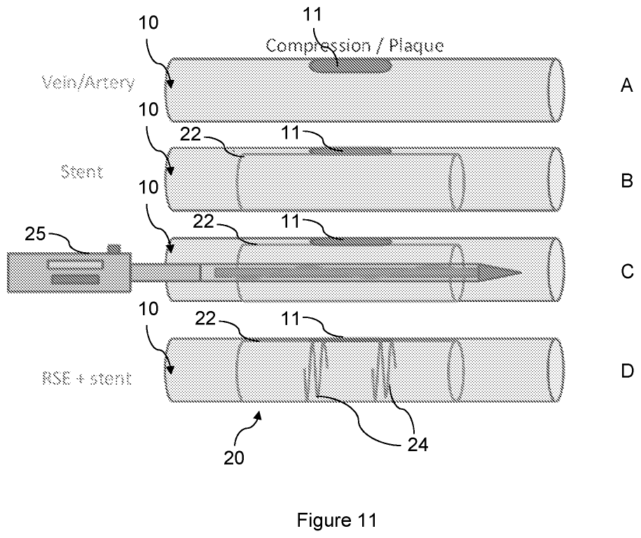

The deployment of the stent system 20, which includes the primary stent 22 and one or more stent elements 24, is depicted in embodiments illustrated in FIGS. 11 to 14. The general process of inserting the system 20 is shown in FIG. 11, while the deployment of the stent elements 24 is provided in FIGS. 12 and 13, and the features used for positioning of the stent elements 24 is depicted in FIG. 14.

In FIG. 11A, a blood vessel 10 is illustrated, which may be either a vein or an artery. The vessel 10 has a region of venous compression or arterial plaque 11 that creates an obstruction to healthy blood flow as well as a sub-optimal aspect ratio of the vessel lumen. For example, the patient may be suffering from May-Thurner Syndrome or DVT. To alleviate the compression/plaque 11, a stent system 20 according to embodiments described herein is provided. The stent system 20 comprises a primary stent 22 and at least two stent elements 24 located therein. In general, the system 20 may comprise a deployment catheter having an over the wire design with a central shaft that can accommodate an IVUS catheter such that IVUA can be used at the same time as deploying the stent element 24. The system has a covered element to allow safe introduction through the hemostasis valve of an introducer, a thumb slide and a locking mechanism. The stent element 24 is packaged in an elongated form, for ease of access and via the thumb slide can be controlled to its deployed expanded form at the desired location. There may be one or more slotted windows along the shaft of the delivery catheter that allow for visualization with IVUS if available for precise positioning of the stent element 24. Once the stent element is deployed in the appropriate position and the operator is satisfied with the position, the device can be released by unlocking the proximal and distal ends from the delivery system. If re-positioning is required, the operator can extend the thumbslide, reposition the delivery system and re-deploy the stent segment 24.

As shown in FIG. 11B, the primary stent 22 is inserted into the vessel 10 and positioned in the region of the compression/plaque 11. As described above, the primary stent 11 may be any suitable stent. To alleviate compression/plaque 11 it is envisaged that the stent is uniformly flexible and of the same diameter along its length. The primary stent 22 is inserted in a conventional way, using a catheter (not shown). Catheters are suitably constructed in a variety of sizes typically ranging from 0.6 mm up to 3.33 mm in diameter (corresponds to French sizes 2 to 10). Guidewires for use with catheters of the invention are typically in the size range of 0.05 mm to about 1 mm (about 0.002 inches to about 0.05 inches). the catheter body is suitably manufactured from plastics or polymeric biocompatible materials known in the technical field, for example, PTFE. In one embodiment of the invention (not shown), the device catheter body may be manufactured from a flexible material so as to enable the device to follow the natural curvature of the lumen of the vessel through which it is travelling.

As shown in FIG. 11C, a stent element deployment device 25 is advanced along the vessel, through the lumen of the vessel 10 and previously deployed primary stent 22. It is envisaged that the deployment device 25 comprises a catheter inserted through a sheath, guided using x-ray or ultrasound equipment. For example, the catheter may be an intravascular ultrasound (IVUS) catheter. The device 25 is described in more detail in relation to FIG. 12.

Once the device 25 is correctly positioned, the stent element 24 is formed within the primary stent 22. Once the stent element 24 is correctly positioned, it is disconnected from the device 25 and the device 25 is removed. As shown in FIG. 11D, in this embodiment two stent elements 24 are placed at either end of the compression/plaque region 11. The stent elements 24 may be any of the elements 24 described in relation to FIGS. 3A to 10, and may be attached to the primary stent 22 using an anchor or engaging mechanism (not shown). In a further embodiment as shown in FIG. 14, a single stent element 24 may be located adjacent to and in correspondence with the occlusive compression 11 so as to bear against the occlusion and restore patency to the primary stent 22.

The steps of forming a coil stent element 224 according to one embodiment of the invention is shown in FIGS. 12A to 12H. It will be appreciated that the depiction of the device in FIGS. 11C, 12A to 12H and 13A to D is foreshortened and not to scale for ease of representation, and that in use the devices conform to the configuration of an elongate percutaneous catheter. The stent element 224 is provided using the deployment device 25. The deployment device 25 comprises a handle 50 at a proximal end of the device 25 attached to a catheter body 52 that extends to a distal end of the device 25. The catheter body 52 may define and encompass a central lumen that extends along the device, or at least a substantial portion of the device. The handle 50 is utilized by the user of the device 25, typically a medical practitioner, to control and manoeuvre the catheter body 52. The catheter body 52 comprises a stent element deployment portion 54 at or in the region of the distal end. The stent element deployment portion 54 comprises a tip portion 56 defining the distal end. An outer sheath 58 covers a cylindrical core, which may be in the form of a mandrel, and which provides a stent element carrier 60. To deliver and deploy the stent element 24, a wire 62 for the stent element 24 is wound around the stent element carrier 60. The wire 62 may be comprised of substantially circular wire, braided wire or in the form of a ribbon. Suitably the wire 62 will be made from a deformable material such as a metal or metal alloy, such as steel or nitinol. The outer sheath 58 is coaxially aligned and configured to cover the stent element carrier 60 and the wire 62 and is further configured to be retracted or withdrawn along the catheter body 52 towards the handle 50 proximally so as to expose the wire 62 so that the stent element 24 can be formed in situ and deployed. A retraction mechanism within the handle, such as a lever or pull wire, may be utilized to mediate the retraction. The stent element carrier 60 comprises two attachment points 64, 66 for connecting the wire 62 to the stent element carrier 60. The attachment points 64, 66 are spaced longitudinally along the carrier 60 so that one of the attachment points 64 is closer to the distal end than the other attachment point 66 which is sited more proximally.

The longitudinal position of one of the attachment points 64 of the stent element carrier 60 may be varied relative to the other, so that the attachment points 64, 66 can be brought closer together to create the stent element 24, as will be described below. The rotational orientation of one of the attachment points 64 may also be varied relative to the other so that the attachment point can rotate about the stent element carrier 60 relative to the other attachment point 66, thereby twisting or untwisting the wire allows the user to apply or release torque and so aid with formation of the stent element 24. At the attachment points 64, 66, a stent element release mechanism is provided configured to releasably connect the wire 62 to the stent element carrier 60 so that the stent element 24 can be detached from the device 25 and deployed. The movement of the attachment points 64, 66 and the release mechanism are controlled at the handle 50 by the user. Release may be effected via a mechanism involving pin release, clamp release or clipping/nipping off of the wire 62. In embodiments where pin release is used the stent element 24 may comprise an aperture at or near the terminus of the wire 62 (as depicted in FIG. 17) for engagement with a retractable pin release mechanism comprised within the carrier 60.

It will be appreciated that user control of the various functionalities described as comprised within the distal end of the device 25 are mediated through control interfaces and mechanisms located within the handle 50. Such interfaces and controls may include, but are not limited to: sliders, levers, screw threads and mechanical or electrical actuators.

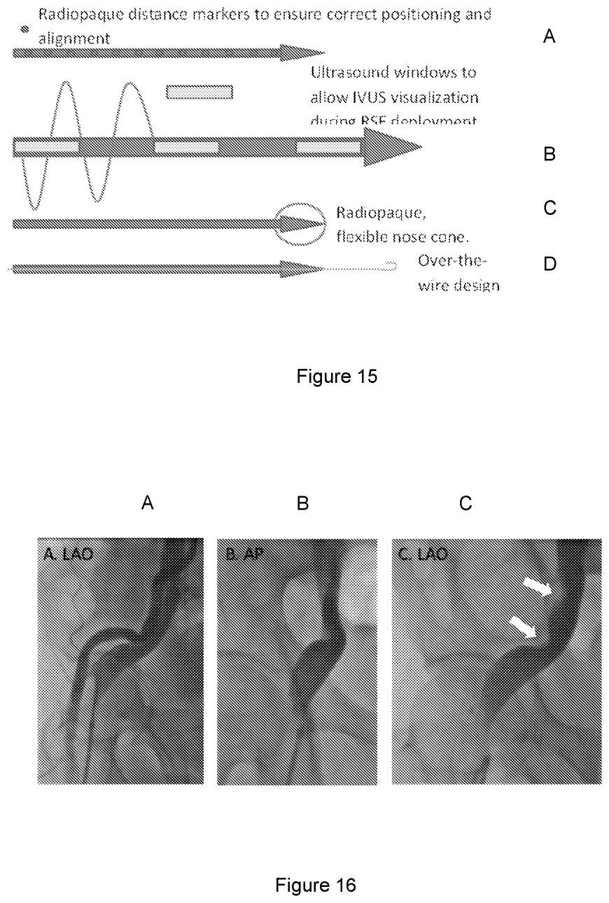

The device 25 may also incorporate one or more means for positioning the catheter 52 and stent element 24. FIGS. 15A to 15D provide various different examples of these positioning mechanisms for aligning the stent element during deployment. As illustrated in FIG. 15A, the catheter may have radiopaque position markers along its length for correct alignment. FIG. 15B illustrates how the catheter may also comprise ultrasound windows to permit IVUS visualization during deployment of the stent elements. Alternatives presented in FIGS. 15C and 15D are respectively that the nose (distal terminus) of the catheter may be radiopaque and flexible and that the catheter may be advanced over a guide wire for correct positioning. In other embodiments, the attachment points of the device may each have a radiopaque marker to provide an indication of the locations of the attachment points relative to one another.

In the method of deployment using the device 25, initially, as can be seen in FIG. 12A, the wire 62 of the unformed stent element 24 is wound around the stent element carrier 60 of the device 25 and covered with the outer sheath 58 while it is delivered to the site for deployment. The outer sheath 58 ensures that the stent element 24 can be delivered without being harmed or without perforating any of the vessels through which it is delivered. The stent element wire 62 is wound around the carrier 60 a predetermined number of times corresponding to how many coils are desired in the stent element 24.

Once at the correct site, the outer sheath 58 is retracted/withdrawn, as shown in FIG. 12B. The ends of the wire 62 are moved radially relative to one another by the attachment points 64, 66 so that the wire 62 untwists and expands radially outwards from the surface of the carrier 60.

The ends of the wire 62 are then brought closer together (i.e. more proximate to each other), as shown in FIGS. 12C, 12D, and 12E, by moving—e.g. slidably or by operation of a screw thread—one attachment point 64 longitudinally along the carrier 60 relative to the other attachment point 66. The radial diameter defined by the wire 62 increases while its distance between the ends decreases. At FIG. 12E, the stent element 24 is fully formed by the wire 62 and at the correct diameter for deployment.

Therefore, when positioning the stent element 24, the device 25 is initially positioned so that the proximal attachment point 66, and therefore the proximal end of the wire 62 and eventually of the stent element, proximal and distal relative to proximal and distal ends of the device 25, is in the correct position as it will be when it is deployed. The formation of the stent element 24 then brings the distal end of the wire 62 at the distal attachment point 64 back towards the proximal end so that stent element 24 is formed at the correct location.

It will be appreciated that this method of forming a stent element permits a stent element of variable diameter to be formed. The diameter of the stent element may be pre-determined and the device configured to create a stent element of that diameter. Alternatively, the user of the device may judge the correct diameter when positioning the device so that the stent element is exactly the correct size for the vessel and primary stent in which the element is being positioned. The diameter can therefore be tailored exactly to the vessel and stent. Conventionally, where the stent had to incorporate the correct properties, rather than a primary stent and stent element, the stent had to be exactly the correct size for the vessel, and choosing this size would be time consuming and would require many different stents of different sizes to be maintained for use. Now, the primary stent may be of a set size, but may be further expanded by the action of the stent element so that the primary stent and the stent element are the correct size and/or aspect ratio at all times.

Once the stent element 24 is formed at FIG. 12E, it will already be in contact with the interior surface of the lumen of the primary stent 22 and therefore should be held in position.

At FIG. 12F, a release button 68 on the handle 50 is operated to release the stent element 24 and, as shown in FIG. 12G, pins are released that were holding the ends of the stent element 24 to the device 25 at the attachment points 64, 66. The stent element 24, shown alone in FIG. 12H, is therefore free of the device 25, which can subsequently be removed leaving the stent element 24 in place within the primary stent 22. This allows the process to be repeated.

FIGS. 13A to 13D show an abridged version of FIGS. 12A to 12H for a stent element having a locking mechanism. The resultant stent element has its ends joined together to avoid any potential damage to the primary stent or vessel, and to improve its strength.

In embodiments where the stent element is formed as an S-ring rather than a coil element, the device may be configured to mount a compressed S-ring around its carrier element, with the outer sheath over the S-ring. The S-ring can subsequently be released once the device is correctly positioned and the outer sheath has been withdrawn, by pin or clamp release or otherwise.

It will be appreciated that alternative radial expansion mechanisms may be implemented such as by introducing the stent element over a radially expandable bladder or balloon catheter device. In such an embodiment the bladder or balloon may be located appropriately in the location for deployment within a primary stent within a vessel and expanded to position the stent element appropriately. Upon deflation of the bladder or balloon the device may be withdrawn from the vessel leaving the stent element in situ.

The above stent system including at least one primary stent and one or more stent elements may be particularly useful in the venous system. For example, the system may be particularly useful at locations of venous obstruction, which includes, at least, venous stenosis, venous congestion, and venous constriction. The stent system described herein may be used in the treatment of MTS, DVT, intraluminal thickening, venous ulcers, venous compression, and/or any other venous or arterial obstruction.

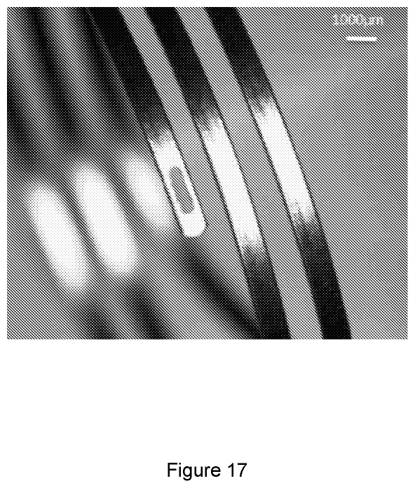

According to one non-limiting example, an individual may have no apparent signs or symptoms of leg swelling but, nevertheless, an obstruction of the veins in the ilio-caval region may be suspected. Normal anatomy in this region sees the vein assume an upward sigmoidal curve from the femoral vein to the inferior-vena cava. In FIG. 16A-C an example of arterial compression of an adjacent underlying vein is observed using contrast fluoroscopy. It would be apparent to the skilled person that relieving the obstruction in this region by implanting a stent with low flexibility and high crush resistance would profoundly alter the local anatomy and may not be in the best interests of the body. For instance, application of a stent with fixed radial force/compression force, would most likely straighten out this region of the vein alleviating compression from the overriding artery. In the longer term this could induce restenosis and intimal hyperplasia resulting in stent failure and more severe venous occlusion. However, according to embodiments of the present invention described in more detail above a more promising solution would involve implanting a highly flexible primary stent to cover this region of the vein and, in so doing, maintain as normal vein positioning and orientation as possible (i.e. not moving or dislocating it). Subsequent positioning in situ of one or more reinforcing stent elements only at the specific points where the compressions are observed (see white arrows in FIG. 16 C) allows for the restoration of luminal patency and normal blood flow.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims. In addition, the above described embodiments may be used in combination unless otherwise indicated.

The invention is further exemplified by way of the following clauses:

1. A stent system comprising:
a primary stent for location in a lumen of a target vessel, the primary stent defining an exterior surface that contacts a vessel wall and an interior surface that faces inwardly;
at least one secondary stent element deployable wholly within the primary stent and configured to engage with the interior surface of the primary stent,

wherein the at least one secondary stent element is configured to apply a chronic outward radial force to the interior surface of the primary stent so as to effect modification of or to resist change to an aspect ratio of the lumen of the target vessel at the location where the secondary stent element is deployed.

2. The stent system of clause 1, wherein the at least one secondary stent element comprises one or more anchors for engaging with the interior surface of the primary stent.

3. The stent system of clause 1 or 2, wherein the at least one secondary stent element is configured to engage the interior surface of the primary stent for modifying the aspect ratio and cross section of the lumen to be substantially circular when deployed.

4. The stent system of any one of clauses 1 to 3, wherein the at least one secondary stent element is configured to apply a substantially uniform chronic outward radial force to the primary stent around its circumference when deployed.

5. The stent system of any previous clause, wherein the at least one secondary stent element has a substantially circular cross-section when deployed.

6. The stent system of any previous clause, wherein the at least one secondary stent element has a substantially elliptical cross-section when deployed.

7. The stent system of any previous clause, wherein the at least one secondary stent element comprises an S ring.

8. The stent system of any previous clause, wherein the at least one secondary stent element comprises coil.

9. The stent system of any previous clause, wherein the system comprises more than one secondary stent element.

10. A stent system for restoring patency to a fully or partially occluded target vessel within the body of a subject, the system comprising:
a primary stent for location in a lumen of the target vessel, the primary stent defining an exterior surface that contacts a vessel wall and an interior surface that faces inwardly;
a plurality of secondary stent elements deployable wholly within the primary stent and configured to engage with the interior surface of the primary stent,

wherein the plurality of secondary stent elements are configured to apply a chronic outward radial force to the interior surface of the primary stent so as to effect modification of an aspect ratio of the lumen of the target vessel at the location where the secondary stent elements are deployed.

11. The stent system of clause 10, wherein the aspect ratio of the lumen of the target vessel is modified to approximate unity in order to restore patency to the fully or partially occluded target vessel.

12. The stent system of clause 10 or 11, wherein the vessel is a vein.

13. The stent system of any one of clauses 10 to 12, wherein the at least one secondary stent element comprises an S ring.

14. The stent system of any one of clauses 10 to 13, wherein the at least one secondary stent element comprises a coil.

15. A percutaneous device for deploying a stent element within a vessel located within an individual subject, the device being of elongate configuration having a proximal end and a distal end, the device comprising:
a handle located at the proximal end for mediating control of the device and deployment of the stent element by a user;
a catheter body that extends to the distal end of the device, the catheter body defining and encompassing a central lumen; and
a stent element carrier located at the distal end of the device, the stent element carrier comprising,
   an elongate, cylindrical core around which at least one wire for forming the stent element is placed, the core comprising a proximal releasable anchor point and a distal releasable anchor point, and wherein the at least one wire extends between and is anchored to the proximal and distal releasable anchor points; and
   a slidable outer sheath capable of being retracted proximally;
wherein at least one of the proximal and distal releasable anchor points is configured to be movable relative to the other along the longitudinal axis of the device.

16. The device of clause 15, wherein the distal releasable anchor point is movable relative to the proximal releasable anchor point, and the position of the proximal releasable anchor point is fixed.

17. The device of clause 15, wherein the proximal releasable anchor point is movable relative to the distal releasable anchor point, and the position of the distal releasable anchor point is fixed.

18. The device of any one of clauses 15 to 17, wherein the stent element carrier is comprised within the central lumen.

19. The device of clause 18, wherein the central lumen extends through the stent element carrier to the distal end.

20. The device of any one of clauses 15 to 19, retraction of the slidable outer sheath is controlled via a retraction mechanism comprised within the handle.

21. The device of any one of clauses 15 to 20, wherein release of the at least one wire from the proximal and distal releasable anchor points is controlled by a release mechanism comprised within the handle.

22. The device of any one of clauses 15 to 21, wherein the proximal and distal releasable anchor points may be rotated relative to each other to apply or release torque to the wire.

23. The device of any one of clauses 15 to 22, wherein the stent element carrier includes a positioning mechanism.

24. The device of clause 23, wherein the positioning mechanism comprises one or more radiopaque markers located along the stent element carrier.

25. The device of clauses 23 or 24, wherein the positioning mechanism comprises one or more ultrasound windows located along the along the stent element carrier.

26. A stent element comprising:
at least one wire formed in a spiral with both ends biased outwardly from a central axis of the spiral;
the outwardly biased ends configured to engage with spaces between the wires of a previously placed braided stent in order to,
(i) anchor the stent element to prevent longitudinal migration and,
(ii) prevent additional circumferential expansion of the stent element and,
(iii) to resist circumferential collapse of the stent element.

27. A method of treating an occlusion of a vessel or duct within the body of a subject, the method comprising:
(a) deploying a primary stent within the occluded vessel at a location that spans the occlusion;
(b) deploying at least one secondary stent element within the primary stent so that the secondary stent element applies a raidial chronic outward force upon the primary stent thereby relieving the occlusion and restoring patency to the vessel or duct.

28. The method of clause 27, wherein the vessel is a vein.

29. The method of clause 28, wherein the vein is within the iliocaval region.

30. The method of any one of clauses 28 or 29, wherein the method is to treat May-Thurner syndrome.

31. The method of any one of clauses 28 or 29, wherein the method is to treat deep vein thrombosis.

32. The method of any one of clauses 28 to 31, wherein more than one secondary stent element is deployed.

33. The method of any one of clauses 28 to 32, wherein deployment of the at least one secondary stent element changes the aspect ratio of a lumen of the vessel at the location of the occlusion to around unity.

1. A stent system comprising:
a primary stent for location in a lumen of a target vessel, the primary stent defining an exterior surface that contacts a vessel wall and an interior surface that faces inwardly;
at least one secondary stent element deployable wholly within the primary stent and configured to engage with the interior surface of the primary stent,
wherein the at least one secondary stent element is configured to apply a chronic outward radial force to the interior surface of the primary stent so as to effect modification of or to resist change to an aspect ratio of the lumen of the target vessel at the location where the secondary stent element is deployed.

2. The stent system of claim 1, wherein the at least one secondary stent element comprises one or more anchors for engaging with the interior surface of the primary stent.

3. The stent system of claim 1, wherein the at least one secondary stent element is configured to engage the interior surface of the primary stent for modifying the aspect ratio and cross section of the lumen to be substantially circular when deployed.

4. The stent system of claim 1, wherein the at least one secondary stent element is configured to apply a substantially uniform chronic outward radial force to the primary stent around its circumference when deployed.

5. The stent system of claim 1, wherein the at least one secondary stent element has a substantially circular cross-section when deployed.

6. The stent system of claim 1, wherein the at least one secondary stent element has a substantially elliptical cross-section when deployed.

7. The stent system of claim 1, wherein the at least one secondary stent element comprises an S ring.

8. The stent system of claim 1, wherein the at least one secondary stent element comprises coil.

9. The stent system of claim 1, wherein the system comprises more than one secondary stent element.

10. A stent system for restoring patency to a fully or partially occluded target vessel within the body of a subject, the system comprising:
a primary stent for location in a lumen of the target vessel, the primary stent defining an exterior surface that contacts a vessel wall and an interior surface that faces inwardly;
a plurality of secondary stent elements deployable wholly within the primary stent and configured to engage with the interior surface of the primary stent,
wherein the plurality of secondary stent elements are configured to apply a chronic outward radial force to the interior surface of the primary stent so as to effect modification of an aspect ratio of the lumen of the target vessel at the location where the secondary stent elements are deployed.

11. The stent system of claim 10, wherein the aspect ratio of the lumen of the target vessel is modified to approximate unity in order to restore patency to the fully or partially occluded target vessel.

12. The stent system of claim 10, wherein the vessel is a vein.

13. The stent system of claim 10, wherein the at least one secondary stent element comprises an S ring.

14. The stent system of claim 10, wherein the at least one secondary stent element comprises coil.

15. A percutaneous device for deploying a stent element within a vessel located within an individual subject, the device being of elongate configuration having a proximal end and a distal end, the device comprising:
a handle located at the proximal end for mediating control of the device and deployment of the stent element by a user;
a catheter body that extends to the distal end of the device, the catheter body defining and encompassing a central lumen; and
a stent element carrier located at the distal end of the device, the stent element carrier comprising,
an elongate, cylindrical core around which at least one wire for forming the stent element is placed, the core comprising a proximal releasable anchor point and a distal releasable anchor point, and wherein the at least one wire extends between and is anchored to the proximal and distal releasable anchor points; and
a slidable outer sheath capable of being retracted proximally;
wherein at least one of the proximal and distal releasable anchor points is configured to be movable relative to the other along the longitudinal axis of the device.

16. The device of claim 15, wherein the distal releasable anchor point is movable relative to the proximal releasable anchor point, and the position of the proximal releasable anchor point is fixed.

17. The device of claim 15, wherein the proximal releasable anchor point is movable relative to the distal releasable anchor point, and the position of the distal releasable anchor point is fixed.

18. The device of claim 15, wherein the stent element carrier is comprised within the central lumen.

19. The device of claim 18, wherein the central lumen extends through the stent element carrier to the distal end.

20. The device of claim 15, retraction of the slidable outer sheath is controlled via a retraction mechanism comprised within the handle.

21. The device of claim 15, wherein release of the at least one wire from the proximal and distal releasable anchor points is controlled by a release mechanism comprised within the handle.

22. The device of claim 15, wherein the proximal and distal releasable anchor points may be rotated relative to each other to apply or release torque to the wire.

23. The device of claim 15, wherein the stent element carrier includes a positioning mechanism.

24. The device of claim 23, wherein the positioning mechanism comprises one or more radiopaque markers located along the stent element carrier.

25. The device of claim 23, wherein the positioning mechanism comprises one or more ultrasound windows located along the along the stent element carrier.

26. A stent element comprising:
at least one wire formed in a spiral with both ends biased outwardly from a central axis of the spiral;
the outwardly biased ends configured to engage with spaces between the wires of a previously placed braided stent in order to,
(i) anchor the stent element to prevent longitudinal migration and,
(ii) prevent additional circumferential expansion of the stent element and,
(iii) to resist circumferential collapse of the stent element.

27. A method of treating an occlusion of a vessel or duct within the body of a subject, the method comprising:
(a) deploying a primary stent within the occluded vessel at a location that spans the occlusion;
(b) deploying at least one secondary stent element within the primary stent so that the secondary stent element applies a raidial chronic outward force upon the primary stent thereby relieving the occlusion and restoring patency to the vessel or duct.

28. The method of claim 27, wherein the vessel is a vein.

29. The method of claim 28, wherein the vein is within the iliocaval region.

30. The method of claim 27, wherein the method is to treat May-Thurner syndrome.

31. The method of claim 27, wherein the method is to treat deep vein thrombosis.

32. The method of claim 27, wherein more than one secondary stent element is deployed.

33. The method of claim 27, wherein deployment of the at least one secondary stent element changes the aspect ratio of a lumen of the vessel at the location of the occlusion to around unity.
